(19) United States
(12) Patent Application Publication
(10) Pub. No.: US 2007/0141724 A1
(43) Pub. Date: Jun. 21, 2007

(54) Solid phase immobilized trifunctional linker

(76) Inventors: Jurgen G. Schmidt, Los Alamos, NM (US); Basil I. Swanson, Los Alamos, NM (US); Clifford J. Unkefer, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 11/590,103

(22) Filed: Oct. 30, 2006

Related U.S. Application Data
(63) Continuation-in-part of application No. 10/629,984, filed on Jul. 29, 2003, now abandoned.

(51) Int. Cl. C40B 40/10; C07K 1/02

(52) U.S. Cl. 436/518; 530/333; 525/54.1

(57) ABSTRACT

The present invention is directed to a method of forming an immobilized chemical moiety, such as a trifunctional chemical moiety that includes membrane-anchoring functionalities, on a solid support for use in automated chemical synthesis of a recognition molecule. The invention is further directed to a method of easily and efficiently synthesizing chemical moieties such as those used for biosensor applications. The invention is further directed to a composition, i.e., a solid support having a trifunctional chemical moiety linked thereto, that can be readily and easily used to generate biological molecules for applications, such as biosensor applications.

### RELATED APPLICATIONS

This application claims the benefit of U.S. application Ser. No. 10/629,984, filed Jul. 29, 2003.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates to immobilization of chemical moieties, such as multifunctional chemical moieties that include membrane-anchoring functionalities, onto a solid resin support for use, e.g., in automated chemical synthesis of a recognition molecule.

### BACKGROUND OF THE INVENTION

Solid phase and combinatorial chemistry are very important in the production and screening of collections or “libraries” of compounds. These libraries are of increasing importance in medicinal chemistry and the discovery of new therapeutic agents. These synthetic methods have been especially developed for the generation of peptides and oligonucleotides.

Generally, the methods used in solid phase and combinatorial chemistry involve immobilizing or capturing the substrate to be modified on a resin or other solid support. Immobilization has the advantage over solution phase chemistry in that purification of the modified substrate is greatly simplified. Additionally, the use of multiple solid supports (e.g., pins, beads, etc.) in a combinatorial approach allows for the production of a large number of diverse compounds, i.e., libraries, in a single operation. The immobilization or capture of the substrate is usually, but not always, accomplished by covalent attachment of the substrate to the resin or other solid support through a linker.

Libraries of compounds produced by combinatorial methods are a powerful tool in the discovery of new materials. Such libraries, which are designed to provide diverse mixtures of compounds, allow for, in combination with high throughput screening, the rapid screening of a large number of a variety of compounds based on a common scaffold. This diversity is a valuable feature of the libraries. Libraries based on a wide variety of scaffolds have been reported. To date, however, solid phase or combinatorial syntheses of diverse libraries of hydrophobic anchor containing-recognition molecules have not been reported.

Recognition molecules, such as peptides, antibodies, oligosaccharides, and oligonucleotides, in general natural and manmade recognition elements, are widely used in a variety of applications that require detection or delivery of a target molecule. Two areas of application are in preparation of complicated molecules for drug delivery and in the development of biosensor technologies.

In the context of drug delivery, particular attention is focused on targeting a drug to a specific site and on efficiency of drug uptake by the targeted cells. The efficiency of drug uptake by cells has been improved by attaching the drug (e.g., a radiolabel) to a hydrophobic chain that facilitates entry of the drug into a cell. Specifically targeting a drug to a particular target cell population can further enhance the efficacy of drug treatment.

Biosensors are devices that detect chemical or biological species with high selectivity on the basis of molecular recognition. Biosensor technology has grown rapidly over the last several years and incorporates technological improvements in a variety of disciplines, including biochemical methodologies (e.g., organic synthesis and molecular biology), and electronics. The potential market for application of biosensor technology is enormous and includes detection and diagnostics in the health care industry and environmental monitoring.

A biosensor device typically incorporates a biological recognition element and a reporter molecule in close proximity or integrated with a signal transducer to provide specific detection of a target molecule (i.e., analyte), such as a protein, bacteria, or virus. Examples of biological recognition elements include peptides (e.g., antibodies, antibody fragments and receptors), oligonucleotides, and oligosaccharides that specifically recognize and bind a target molecule. Examples of reporter molecules include fluorophores, isotopic labels, magnetic materials, or other chemical and biochemical entities or labels that yield an externally measurable output signal that can be correlated or assigned with a specific binding event. A signal transducer is generally a device that transforms the binding event between the target molecule and the biological recognition molecule into a measurable signal, such as a fluorescent signal. In general, biosensors are devices that detect (i.e., “sense”) and/or quantify molecules of interest. Such detection or sensing occurs when there is an interaction between the target molecule and the biological recognition molecule (e.g., an antibody, receptor, or DNA strand).

Biosensor platform technologies based on optical detection of analytes by fluorescence of a reporter molecule have been described. One type of biosensor includes recognition molecules, such as receptor molecules or antibody fragments that are anchored to and freely mobile in a lipid bi-layer membrane. The recognition molecule is typically anchored in a bi-layer membrane by a hydrophobic anchoring moiety. The formation of recognition molecule/bi-layer membrane complexes generally requires several chemical and molecular reactions, such as formation of a lipid bi-layer and synthesis of a recognition molecule. U.S. patent application Ser. No. 10/104,158, by Schmidt et al., entitled, “Generic Membrane Anchoring System,” describes a trifunctional chemical moiety that includes a central core (i.e., “trifunctional” core) comprised of an amino acid or analog thereof with three chemically reactive sites for attaching different functional molecules, such as a recognition molecule, a reporter molecule, and a membrane anchoring molecule for use in applications, such as a lipid bi-layer biosensor.

FIG. 1 illustrates an example of a structure of a trifunctional chemical moiety 100 described in U.S. patent application Ser. No. 10/104,158. Trifunctional chemical moiety 100 includes a trifunctional linker core 105, a membrane anchor 110, a reporter molecule 120, and a spacer 130. Core 105 is typically an amino acid or amino acid analog, such as, but not limited to, cysteine, glutamic acid or lysine. Spacer 130 extends from core 105, typically from an amino acid side-chain and culminates in a chemically reactive site. The chemically reactive site on spacer 130 is generally used to couple a recognition molecule, such as a peptide, with core 105. Spacer 130 is typically of sufficient length to provide a spatial orientation of a functional molecule, such as a recognition molecule, away from the membrane surface. Exemplary spacers include materials such as a polyalkylene glycol, e.g., polyethylene glycol (PEG) or polypropylene glycol (PPG).

Reporter molecule 120 is typically any chemical or biochemical entity or label that yields an externally measurable output signal that can be correlated or assigned with a specific binding event, such as fluorophores, isotopic labels, or magnetic materials.

Membrane anchor 110 provides mobile attachment of the trifunctional chemical moiety 100 (including core 105, reporter molecule 120, and spacer 130) to a fluid surface of a membrane. Membrane anchor 110 is typically a hydrophobic group and can be any anchoring group that contains alkyl, alkenyl-, alkynyl and polyaromatic chains of carbon containing from about 4 to 30 carbons.

Synthesis of a trifunctional chemical moiety 100 typically uses standard peptide chemistry methods, such as activated esters or in situ activation, to covalently attach a biological recognition molecule onto the chemically reactive site at the terminus of spacer 130 upon core 105. Such a recognition molecule can be, e.g., a peptide or oligonucleotide. The synthesis of trifunctional chemical moiety 100 and attachment of a recognition element are typically sequential, requiring multiple conventional solution-based chemical reactions and purification steps that are often tedious to perform. Thus, there exists a need for a method of easily and efficiently providing the biological recognition portion of such trifunctional chemical moieties, such as membrane anchored recognition sites for use in biosensor applications.

In biosensor applications, a recognition molecule determines the sensitivity and specificity of detecting a target molecule in a sample. In general, a recognition molecule is synthesized in solution-based chemical or molecular reactions prior to integrating the recognition molecule to a biosensor platform technology. Further, in membrane-based biosensor platforms, the long alkyl chains of the membrane anchors used with the recognition element are sparingly soluble in aqueous solutions and often form vesicles or micelles that sequester chemically reactive sites, making them unavailable for subsequent coupling reactions. Solution-based synthesis reactions also require defined reaction volumes and numerous purification steps to remove excess reagents and by-products prior to subsequent reactions.

As an alternative, protocols using solid-phase synthesis of membrane anchor containing-biomolecule conjugates may provide a relatively simple, rapid, and automated means to synthesize a recognition molecule. Solid-phase synthesis typically uses a resin that is insoluble in the solvents used for synthesis and provides a simple and rapid means to wash away excess reagents and by-products. There exists a need for a method of covalently attaching a membrane anchor, such as a multifunctional chemical moiety, to a solid support for automated, sequential or combinatorial syntheses of an attached recognition molecule thereon for biosensor applications. Such an attachment to a solid support may be done in a reversible manner.

Biosensor technology incorporates technologies from a variety of disciplines, including organic chemistry and molecular biology. These different technologies are typically complex and require a high level of expertise in a variety of disciplines to successfully develop a biosensor. For example, synthesis of a recognition molecule is typically performed by a chemist with significant knowledge of chemical synthesis reactions for peptides, oligonucleotides, or oligosaccharides. Thus, there exists a need for a starting resin, such as a multifunctional chemical moiety linked to a solid resin support, that will provide non-chemists a means to generate biological molecules for biosensor applications, using standard automated synthesizers.

It is therefore an object of the present invention to provide a method of easily and efficiently providing immobilized chemical moieties, such as membrane-anchored moieties for use in applications, such as a biosensor application.

It is another object of this invention to provide a method of covalently attaching a multifunctional chemical membrane-anchoring moiety, to a solid support for automated synthesis of an attached recognition molecule thereon for use in applications, such as for a biosensor application. The attachment can be reversible in some embodiments.

It is yet another object of this invention to provide a starting resin, such as a multifunctional chemical moiety linked to a solid resin support, that will provide non-chemists a means to generate biological molecules for applications, such as a biosensor application.

It is yet another object of this invention to provide a starting resin, such as a multifunctional chemical moiety linked to a solid resin support, that will provide a facile entry to molecular diverse biological molecules in combinatorial fashion for applications, such as a membrane-based assay.

### SUMMARY OF THE INVENTION

In accordance with the purposes of the present invention, as embodied and broadly described herein, the present invention provides a composition including a solid resin support having a trifunctional chemical moiety covalently attached thereto at a resin attachment site, the trifunctional chemical moiety including one or more hydrophobic membrane anchoring groups as one functionality thereon, the hydrophobic membrane anchoring groups containing from 4 to 30 carbon atoms with the proviso that the hydrophobic membrane anchoring groups are not directly attached to the solid resin support.

In a particular embodiment of the present invention, the trifunctional chemical moiety includes an amino acid derivative including alkyl side chains as the one or more hydrophobic membrane anchoring groups thereon, a reactive group from the group of amino, hydroxyl, carboxyl and sulfhydryl thereon and a reactive arm group from the group of amino, carboxyl and sulfhydryl covalently attached thereto the resin attachment site.

The present invention further provides a method of solid phase peptide synthesis including reacting a trifunctional chemical moiety including one or more alkyl side chains having from 4 to 30 carbons as hydrophobic membrane anchoring groups thereon, a reactive group from the group of amino, hydroxyl, carboxyl and sulfhydryl thereon and a reactive arm group from the group of amino, carboxyl and sulfhydryl thereon with a solid resin support having a reactive site thereon to form a bound multifunctional chemical moiety-solid resin support composite, and, reacting the bound multifunctional chemical moiety-solid resin support composite in a solid phase synthesis process.

The present invention further provides method of covalently attaching a trifunctional chemical moiety to a solid resin support including reacting a trifunctional chemical moiety including an amino acid derivative including one or more alkyl side chains having from 4 to 30 carbons as hydrophobic membrane anchoring groups thereon, a reactive group from the group of amino, hydroxyl, carboxyl and sulfhydryl thereon and a reactive arm group from the group of amino, carboxyl and sulfhydryl thereon with a solid resin support having a reactive site thereon to form a bound trifunctional chemical moiety-solid resin support composite.

### BRIEF DESCRIPTION OF THE DRAWINGS

FIG. 1 shows a structure of a trifunctional chemical moiety as shown in pending U.S. patent application Ser. No. 10/104,158 by Schmidt et al., for “Generic Membrane Anchoring System” filed on Mar. 21, 2002.

FIGS. 2(a)-(e) show structures of immobilized multifunctional chemical moieties in accordance with the present invention.

FIG. 3 shows a method of immobilizing a multifunctional chemical moiety on a solid substrate in accordance with the present invention.

FIG. 4 illustrates a method of using an immobilized multifunctional chemical moiety for automated chemical synthesis.

FIG. 5 illustrates synthesis of a model peptide in accordance with the present invention.

FIGS. 6(a) and (b) show an ABI conductivity trace and MALDI-MS for a peptide “VPPYFTLMYGGGGK” synthesized on a resin (solid support) immobilized membrane anchor.

### DETAILED DESCRIPTION

The present invention provides a method of forming a multifunctional chemical moiety, e.g., a trifunctional chemical moiety, on a solid-phase support for use in automated chemical synthesis. The method provides advantages in processing steps, such as using combinations of chemicals to attach functional molecule libraries and process advantages intrinsic to solid phase methods, such as facilitated washing and purification. The present invention further provides a composition, i.e., solid phase linked building block for the rapid synthesis of generic membrane anchoring linker systems of the general formula: (Res)(Cg)(mA) where Res is a solid support group, mA is an anchoring group, Cg is a trifunctional core. These linker systems can be pre-assembled containing any suitable membrane-anchoring unit and attached at an amine, sulfhydryl, hydroxyl or carboxyl of an amino acid onto solid phase supports. The anchoring arm, when suitably modified by, e.g., dimethoxytrityl, Fmoc (fluorenylcarbamate) and the like, can then be used in solution and solid phase syntheses of the binding unit, including combinatorial libraries of, e.g., peptides, carbohydrates, nucleosides, and their analogs. This synthetic approach has the further advantage that reporter groups, which are often expensive and labile in chemical transformations, are attached in the final step after product recovery from the resin, with the choice of an amine, carboxyl, hydroxyl or sulfhydryl released on deprotection thus allowing chemoselective post-synthetic modifications in the presence of a wide range of other product functionalities.

The present invention can further allow preparation of chemical moieties including a recognition functionality, a reporter functionality and an anchoring functionality, such an anchoring functionality allowing for attachment of such chemical moieties to a fluid surface of a membrane.

“Amino acid” refers to any of the naturally occurring amino acids, as well as optical isomers (enantiomers and diastereomers), mimetics, synthetic analogs and derivatives thereof. α-Amino acids comprise a carbon atom to which is bonded an amino group, a carboxyl group, a hydrogen atom, and a distinctive group referred to as a “side chain.” Naturally occurring amino acids are those encoded by the genetic code, as well as those amino acids that are later modified, e.g., hydroxyproline, γ-carboxyglutamate, and O-phosphoserine. α-Amino acids also comprise a carbon atom to which is bonded an amino group, a carboxyl group, a sulfhydryl group and two distinctive groups (which can be the same group or can be different groups), in which case the amino acid has two side chains. The side chains of naturally occurring amino acids are well known in the art and include, for example, hydrogen (e.g., as in glycine), alkyl (e.g., as in alanine, valine, leucine, isoleucine), substituted alkyl (e.g., as in threonine, serine, methionine, cysteine, aspartic acid, asparagine, glutamic acid, glutamine, arginine, and lysine), arylalkyl (e.g., as in phenylalanine), substituted arylalkyl (e.g., as in tyrosine), and heteroarylalkyl (e.g., as in histidine and tryptophan). See, e.g., Harper et al. (1977) Review of Physiological Chemistry, 16th Ed., Lange Medical Publications, pp. 21-24. One of skill in the art will appreciate that the term “amino acid” also includes β- α-, δ-, and Ω-amino acids, and the like, and α-imino acids such as proline. As used herein, “amino acids” includes proline. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Non-naturally occurring amino acids are known in the art, as set forth in, for example, Williams (ed.), Synthesis of Optically Active α-Amino Acids, Pergamon Press, 1989; Evans et al. (1990) J. Amer. Chem. Soc., 112:4011-4030; Pu et al. (1991) J. Amer. Chem. Soc. 56:1280-1283; and Williams et al. (1991) J. Amer. Chem. Soc. 113:9276-9286.

“Protecting group” refers to a chemical group that exhibits at least one of the following characteristics: 1) reacts selectively with the desired functionality in good yield to give a protected reactive group or functionality that is stable to the reactions for which protection is desired; 2) is selectively removable from the protected substrate to yield the desired reactive functionality; and 3) is removable in good yield by reagents compatible with the other functional groups of the trifunctional chemical moiety. Examples of suitable protecting groups can be found in Greene et al. Protective Groups in Organic Synthesis, 2nd Ed., John Wiley & Sons, Inc., New York, 1991. Suitable terminal amino protecting groups include benzyloxycarbonyl (CBz), t-butyloxycarbonyl (Boc), t-butyldimethylsilyl (TBDIMS), 9-fluorenylmethoxycarbonyl (Fmoc), allyloxycarbonyl (Alloc), biphenyloxycarbonyl (Bpoc), and triphenylmethyl (trityl) or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Suitable hydroxylprotecting groups include t-butyl, TBDIMS, photolabile protecting groups (such as nitroveratryl oxymethyl ether (Nvom)), Mom (methoxy methyl ether), and Mem (methoxy ethoxy methyl ether). Suitable carboxyl protecting groups can include straight or branched chain (C₁ to C₁₂) alkyl groups (e.g., isopropyl, tert-butyl and the like). “Protected derivative” of a compound is used to refer to a compound, which has been protected with a protecting group, such as those described above. Preferred thiol protecting groups include, but are not limited to, trityl (Trt), p-methoxytrityl (Mmt), p-methyltrityl (Mtt), acetamidomethyl (Acm), benzyl (Bzl), t-butyl (tBu), t-butylthio (tButhio), and p-methoxybenzyl (pMeOBzl).

Common solvents used during the syntheses include N,N-dimethylformamide (DMF), N-methylpyrrolidinone (NMP), 1,2-dimethoxyethane (DME), dichloromethane (DCM), and dimethylacetamide (DMA).

Solid phase synthetic methods allow for rapid and automated access to oligomeric nucleosides and peptides. Solid phase syntheses recently became a widely used platform for combinatorial exploitation of highly diverse product libraries in sequential and parallel fashion. Immobilizing a membrane-anchoring unit provides rapid and automated access to membrane-anchored oligomers as peptides, nucleosides and analogs as well as combinatorial libraries of membrane anchoring products. The data from the examples exemplify the use of these immobilized membrane anchors in peptide syntheses on an Applied Biosystems ABI 433 peptide synthesizer. The present process may be readily adapted to solid phase syntheses practiced as state of the art in combinatorial chemistry for a wide variety of chemical platforms, the use of, e.g., lysine provides amine-; the use of serine and homoserine provide hydroxyl-; the use of glutamic and aspartic acid provide carboxyl; the use of cysteine provides sulthydryl-sites for the initial attachment site. This diversity may be readily extended using other amino acids and analogs and allow a highly versatile platform to accommodate diverse chemical methods to obtain molecular libraries derivatized with hydrophobic end groups. Such a platform can be further suited to generate chemical structures with repeating alkyl-derivatized amino acids in a peptide, peptidic oligomer or heteromeric polymers.

Small peptides can be readily prepared by automated solid phase peptide synthesis (Merrifield et al., Biochemistry 21:5020-5031, 1982; Houghten, Proc. Natl. Acad. Sci. USA 82:5131-5135, 1985; Lin, et al., Biochemistry 27:5640-5645, 1988) using any one of a number of well known, commercially available automated synthesizers, such the Applied Biosystems ABI 433A peptide synthesizer.

The present invention presents the following advantages. First, the process provides rapid access to membrane-anchored chemical moieties containing oligos. Further, the process provides a means for conducting combinatorial syntheses of membrane-anchoring moieties. Further, it is known that C-terminus could be modified without losing activity, so C-terminus modification is provided. The reaction scheme allows the flexibility to conduct N to C termini syntheses in addition to the more common C—N direction. Next, high hydrophobic assembled recognition to make solubility advantage of the process.

The preparation process according to the present invention can be carried out on a solid phase in order to achieve a process implementation, which is as economical as possible. In this case, an amine, carboxyl, hydroxyl or sulfhydryl residue can be bonded to any solid resin support or solid phase conventionally used for reactions of this type. Solid resin supports which can be used may consist of a large number of materials as long as they are compatible with the chemistry used and with the attachment of the multifunctional chemical moiety to the particular solid resin support. Examples of suitable solid resin supports include materials from the group of ceramics, glass, latex, crosslinked polystyrenes, crosslinked polyacrylamides or other resins, natural polymers, gold, colloidal metal particles, silica gels, aerogels or hydrogels. It is also possible, where appropriate, for mixtures of different materials to be used. According to the invention, the solid phase used is particularly preferably a polystyrene resin and in particular commercially available Wang polystyrene resin, e.g, a p-nitrophenyl carbonate-Wang resin. The above reactions and their implementation are well known to the person skilled in the art and are described, for example, by Wang et al., J. Am. Chem. Soc., 1973, 95, 1328-1333.

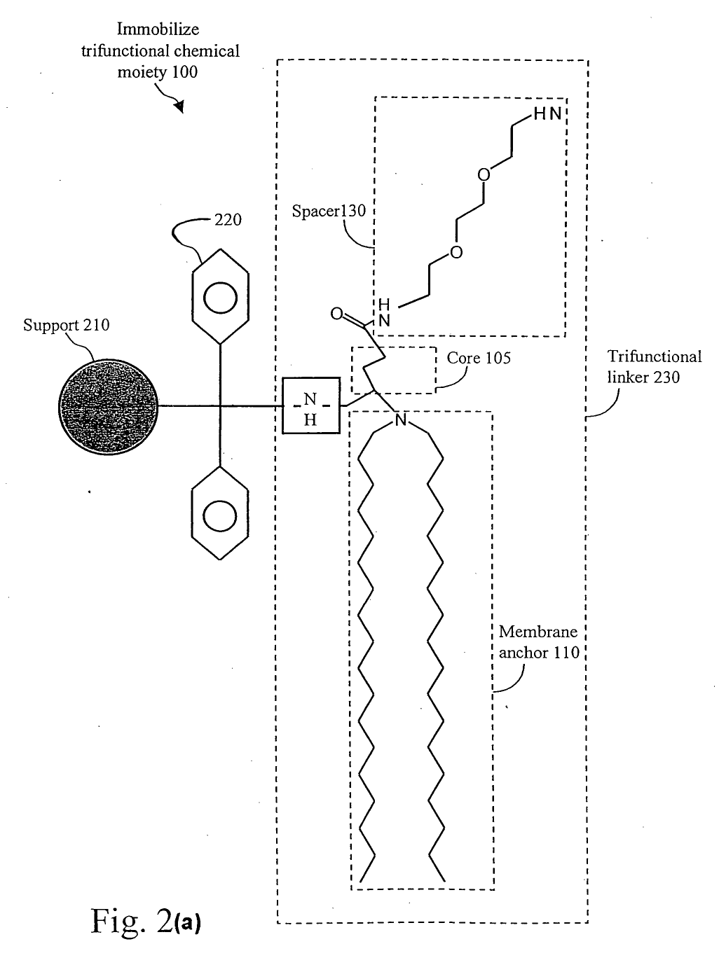

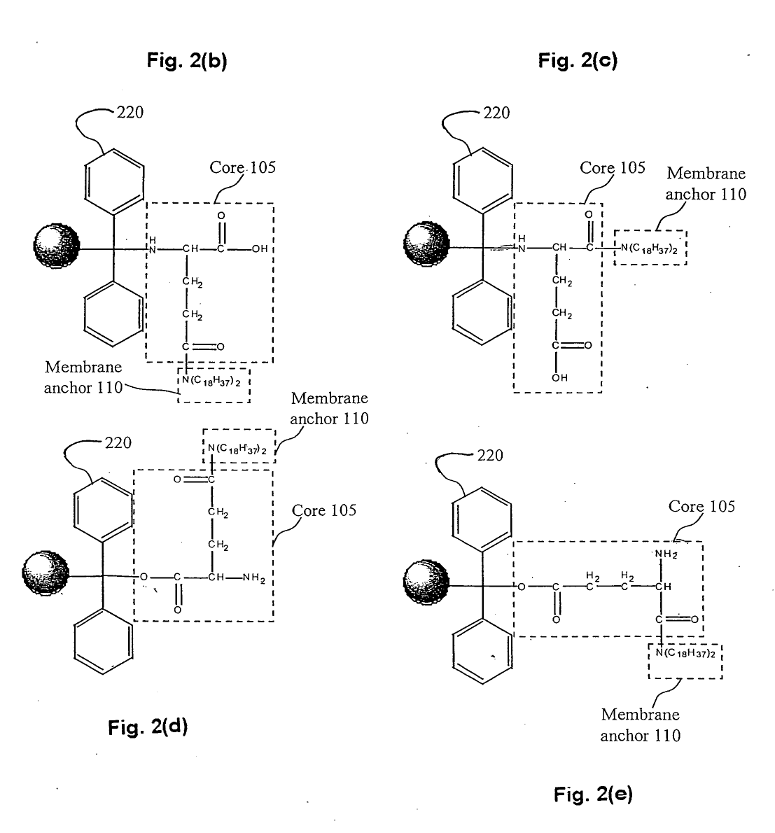

FIG. 2(a) illustrates a typical immobilized trifunctional chemical moiety 200. Immobilized trifunctional chemical moiety 200 includes a solid support 210, a functional linking group 220, and a trifunctional linker 230.

Solid support 210 is typically any solid support material that has a suitable reactive site. For example, in automated peptide synthesis, solid support 210 is typically a polystyrene bead suspension with a size distribution of approximately 90±27 microns and appropriate swelling characteristics in solvents, such as dimethylformamide (DMF), that are commonly used in peptide synthesis reactions.

Reactive site 220 provides chemical functionality for the attachment of solid support 210 to the carboxyl or amino end of core 105 in trifunctional linker 230. Solid support 210 and reactive site 220 are any commercially available solid-support resins that provide specific coupling and removal chemistries. For example, solid support 210 may be a polystyrene bead derivatized with a trityl chloride reactive site 220, a chemical group commonly used in automated peptide synthesis reactions. Solid-support resins are selected based on the chemical synthesis reactions that are used to synthesize a recognition molecule. Core 105 provides flexibility in selecting a solid-support resin from a large number of commercially available resins typically used in automated chemical synthesis. Solid support 210 is insoluble in the solvents used for conventional synthesis reactions, providing relatively simple and rapid removal of excess reagents and by-products.

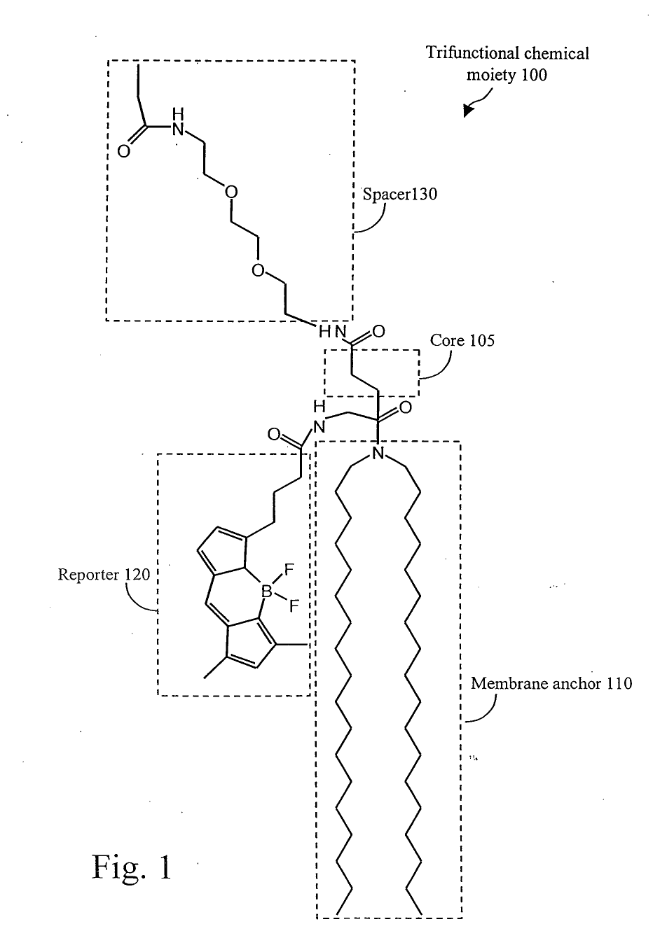

Trifunctional linker 230 includes core 105, membrane anchor 110, and spacer 130, which are described in reference to FIG. 1. Membrane anchor 110 provides mobile attachment of the trifunctional chemical moiety 100 (including core 105, reporter molecule 120, and spacer 130) to a fluid surface of a membrane. Membrane anchor 110 is typically a hydrophobic group and can be any anchoring group that contains alkyl, alkenyl-, alkynyl and polyaromatic chains of carbon containing from about 4 to 30 carbons, more preferably from about 6 to 18 carbons. Preferably, the membrane anchor 110 is an alkyl group containing from about 4 to 30 carbons, more preferably from about 6 to 18 carbons. One exemplary membrane anchor 110 is illustrated in FIG. 2(a) and consists of dialkylamino—(e.g. dioctadecylamine).

The amino end of spacer 130 is typically protected with a blocking group, such as a fluorenylmethoxycarbonyl (Fmoc). Prior to automated chemical synthesis reactions, the blocking group is removed using standard protocols.

Reactive site 220 is chemically attached to trifunctional linker 230 using standard coupling chemistry. For example, a free α-carboxylic acid of core 105, such as a glutamic acid, may be derivatized to contain a cesium (Cs) ester, which chemically reacts with reactive site 220, e.g., a trityl chloride functionality.

FIGS. 2(b)-2(e) illustrate four possible configurations for attachment of linking group 220 and membrane anchor 110 to core 105. In these embodiments, core 105 is a glutamic acid residue. FIG. 2(b) shows reactive site 220 attached to core 105 at the α-amine of glutamic acid and membrane anchor 110 attached to core 105 at the γ-carboxyl of glutamic acid. FIG. 2(c) shows reactive site 220 attached to core 105 at the α-amine of glutamic acid and membrane anchor 110 attached to core 105 at the α-carboxyl of glutamic acid. FIG. 2(d) shows reactive site 220 attached to core 105 at the α-carboxyl of glutamic acid and membrane anchor 110 attached to core 105 at the γ-carboxyl of glutamic acid. FIG. 2(e) shows reactive site 220 attached to core 105 at the γ-carboxyl of glutamic acid and membrane anchor 110 attached to core 105 at the α-carboxyl of glutamic acid.

The arrangements in FIGS. 2(d) and (e) provide a free amine of amino acid core 105 which is available for peptide synthesis. As standard peptide syntheses proceed from the carboxyl terminus to the amino terminus, the arrangements in FIGS. 2(b) and (c) can be used directly only for reverse directional peptide syntheses.

FIG. 2(f) illustrates a preferred use of configurations A and B. Spacer 130, such as a polyethylene glycol spacer, is attached to core 105. Spacer 130 provides a free amine, which is available for standard peptide syntheses.

In an alternative embodiment, core 105 is a lysine amino acid that provides attachment at either the α- or ε-amine of core 105. A preferred use depends on the envisioned characteristics of a final product as an attachment at the side chain of lysine provides an additional four-carbon spacer for attachment of a functional molecule, e.g. a fluorophore.

A further alternative embodiment can use cysteine as the core attached by either amine or sulfhydryl to the resin. The sulfhydryl attachment provides a free thiol on deprotection of the final product, which allows for chemoselective post-synthetic derivatization by sulfhydryl specific reagents.

The coupling chemistry used to covalently attach reactive site 220 to trifunctional linker 230 is dependent on the structure of core 105. Core 105 provides flexibility in selecting linking chemistry for attaching trifunctional linker 230 to a resin. Core 105 also provides flexibility in the direction of chemical synthesis of a recognition molecule such as a peptide, i.e., from carboxyl end to amino end or from amino end to carboxyl end.

In an alternative embodiment of the present invention, core 105 may be a lysine amino acid, which chemically reacts through one of its amino termini with linking group 220, such as a trityl chloride group.

In another alternative embodiment of the present invention, solid support 210, may be a magnetic bead, such as a magnetic bead coated with polystyrene, for chemical attachment of trifunctional linker 230 for use in biosensor applications based on flow cytometry.

Immobilized trifunctional chemical moiety 200 provides solid-phase material for automated chemical synthesis reactions, such as peptide, oligonucleotide, oligonucleotide-peptide analog, or oligosaccharide synthesis, using standard automated synthesis protocols. Immobilized trifunctional chemical moiety 200 provides a starting resin, such as a trifunctional chemical moiety linked to a solid support that may be used by non-chemists to generate biological molecules for membrane-based applications such as biosensors.

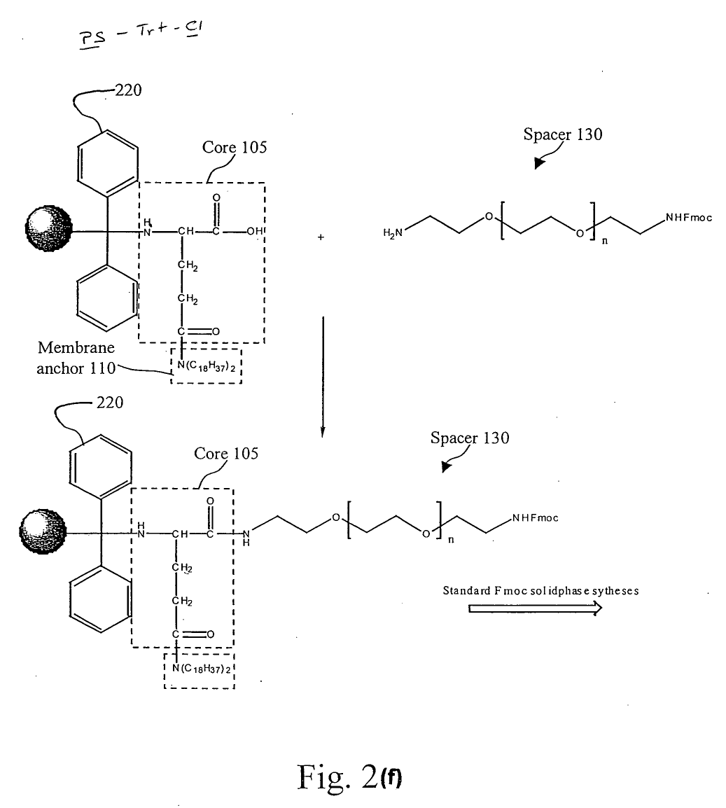

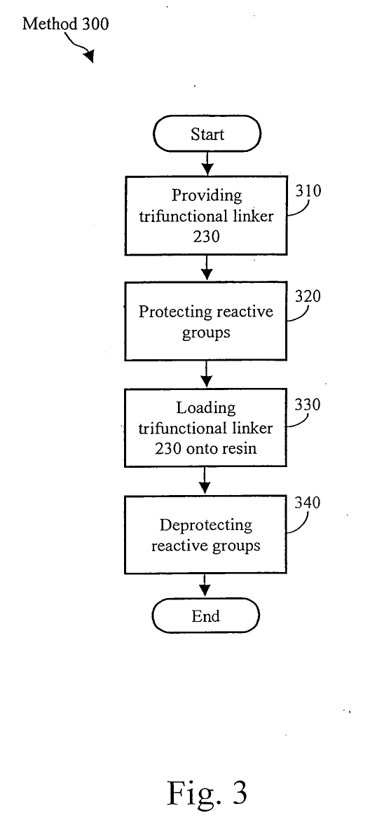

FIG. 3 illustrates a method 300 of forming solid-phase immobilized trifunctional chemical moiety 200. Immobilized trifunctional chemical moiety 200 is typically used in standard automated synthesis reactions to generate recognition molecules, such as peptides, oligonucleotides, or oligosaccharides that are used as in biosensor applications. Method 300 generally includes the steps of: step 310 (providing a trifunctional linker); step 320 (protecting the reactive group); step 330 (loading the trifunctional linker onto a resin); and, step 340 (deprotecting the reactive group).

In initial step 310, trifunctional linker 230 is provided. Trifunctional linker 230 is synthesized as disclosed in U.S. patent application Ser. No. 10/104,158.

In next step 320, a reactive amino group on spacer 130 is protected with a blocking group, such as Fmoc, using standard blocking chemistry. Prior to automated chemical synthesis reactions, the blocking group is removed using standard protocols.

In next step 330, a resin that includes solid support 210 and reactive site 220 is loaded with excess derivatized trifunctional linker 230 using standard resin-loading protocols. For example, a free α-carboxylic acid of core 105, such as a glutamic acid core, is derivatized to become the cesium salt, which then chemically reacts with the solid phase resin under standard loading procedures for trityl resins 220.

In next step 340, the Fmoc protective group is removed from spacer 130 using conventional cleavage chemistry. The solution concentration of liberated Fmoc chemical group is typically measured by ultraviolet spectroscopy and is used to determine the efficiency of resin loading. The efficiency of resin loading using method 300 is typically within the maximum range for a commercially available resin. Immobilized trifunctional chemical moiety 200 is ready for use in automated synthesis reactions for synthesis of recognition molecules, such as peptides, oligonucleotides, or oligosaccharides. Method 300 ends.

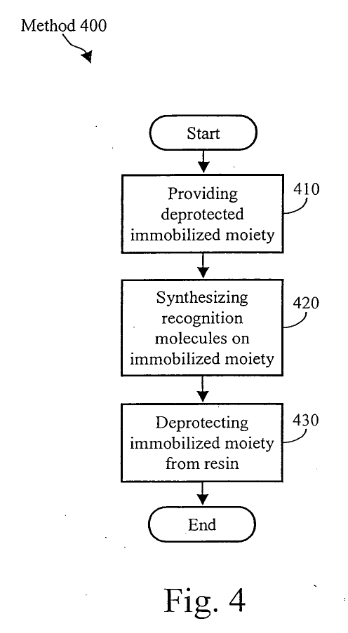

FIG. 4 illustrates a method 400 of using immobilized trifunctional chemical moiety 200. Method 400 generally includes the steps of: step 410 (providing a deprotected immobilized moiety); step 420 (synthesizing recognition molecules); and, step 430 (deprotecting an immobilized moiety from the resin).

In initial step 410, immobilized trifunctional chemical moiety 200 is provided. Immobilized trifunctional chemical moiety 200 is formed as described in greater detail in method 300.

In next step 420, recognition molecules, such as peptides, oligonucleotides, or oligosaccharides, are synthesized on spacer 130 of immobilized trifunctional chemical moiety 200 using standard automated solid-phase synthesis chemistry and protocols.

In next step 430, trifunctional linker 230, including the newly synthesized recognition molecule on spacer 130, is deprotected from solid support 210 by conventional deprotection reactions that cleave the covalent attachment of trifunctional linker 230 to reactive site 220. The deprotection chemistry is generally determined by resin used in the reaction. For example, trifunctional linker 230 immobilized on a polystyrene bead through a trityl chloride reactive site 220 is cleaved from reactive site 220 using trifluoroacetic acid (TFA). In this example, the deprotecting reaction regenerates the carboxyl or amine groups, providing reactive sites for subsequent chemical reactions, such as chemically coupling a reporter molecule or other structure to trifunctional linker 230. Hyperacid sensitive resins such as 2-chloro-trityl allow the deprotection to be conducted under mild deprotection conditions, which leave the sidechain protecting groups of, e.g., peptides, attached to the product. This allows for chemoselective derivization of the amino and carboxyl end before final sidechain deprotection of the products.

Trifunctional linker 230 now includes a recognition molecule attached to spacer 130 that is suitable for biosensor applications.

In yet another alternative embodiment of the present invention, the deprotected site on core 105 previously used to couple trifunctional linker 230 to reactive site 220 may be used to attach a functional molecule, such as a reporter molecule, a second anchoring molecule, or a second recognition molecule.

In yet another alternative embodiment of the present invention, the deprotected site on core 105 previously used to couple trifunctional linker 230 to reactive site 220 may be used to attach a physical entity, such as a derivatized gold or magnetic bead.

In yet another alternative embodiment of the present invention, trifunctional chemical moiety 200, method 300, and method 400 can be used to provide a trifunctional chemical membrane-anchoring moiety on a solid support for automated synthesis of a recognition molecule for use in applications, such as preparation of complex molecules for drug delivery.

In the present specification, amino acid residues are represented using abbreviations, as indicated below, approved by IUPAC-IUB Commission on Biochemical Nomenclature (CBN). With respect to amino acids and the like having isomers, those which are represented by the following abbreviations are of either L-form or D-form. Further, the left and right ends of an amino acid sequence of a peptide are, respectively, the N- and C-termini unless otherwise specified: A or Ala: alanine residue; D or Asp: aspartic acid residue; E or Glu: glutamic acid residue; F or Phe: phenylalanine residue; G or Gly: glycine residue; H or His: histidine residue; I or Ile: isoleucine residue; K or Lys: lysine residue; L or Leu: leucine residue; M or Met: methionine residue; N or Asn: asparagine residue; P or Pro: proline residue; Q or Gln: glutamine residue; R or Arg: arginine residue; S or Ser: serine residue; T or Thr: threonine residue; V or Val: valine residue; W or Trp: tryptophan residue; Y or Tyr: tyrosine residue; and C or Cys: cysteine residue.

All publications, patents, and patent applications cited in this specification are herein incorporated by reference as if each individual publication or patent application were specifically and individually indicated to be incorporated by reference.

The present invention is more particularly described in the following examples which are intended as illustrative only, since numerous modifications and variations will be apparent to those skilled in the art.

The additional following abbreviations are used throughout the following examples.

DIPEA: diisoproylethylamine

HPLC: high performance liquid chromatography

M: molar

MALDI: matrix assisted laser desorption ionization

MS: mass spectrometry

NMR: nuclear magnetic resonance spectrometry

PEGₙ: polyethylene glycol, n indicates the length in ethylene glycol units

RP: reversed phase chromatography

TOF: time of flight

All solvents and reagents were purchased from Fisher Chemical Co. or Sigma-Aldrich (both Aldrich and Fluka brands) and distilled where indicated as dry over suitable drying reagents. Peptide synthesis reagents and solvents are ABI or Fisher peptide syntheses grade. Dioctadecylamine, bisamino-PEG, and PEGN were purchased from Sigma-Aldrich (Fluka brand). Activated and/or protected amino acids and derivatives were purchased from CAL Biosciences, Inc. (NovaBiochem™ brand of products) or AdvancedChemTech, Inc. and used without further purification. Fluorophores such as BODIPY® dyes were obtained from Molecular Probes, Inc. Peptides and peptide syntheses were conducted on an ABI 433A in “0.25 mmol FastMOC Ω prev.peak”. For column chromatography EM Silica gel-60 was used, HPLC-separations were obtained on Varian Prostar equipment with Alltech columns specified in the experimental procedure. NMR were measured on a Bruker Advance 300 or 500 MHz instrument, solvents are specified in the experimental procedures, calibration on NMR solvent as internal standard. MALDI-TOF MS were obtained on a Perseptive Biosystem Voyager using α-cyano-4-hydoxycinnamic acid as matrix; mass results are calibrated to the closest mass match peptide, either Angiotensin or Insulin.

### EXAMPLE 1

Para-nitrophenyl carbonate-Wang resin (NovaBiochem, 0.92 mmol/g; 870 mg; 0.8 mmol) were pre-swollen in 20 mL of DMF, spun down in centrifuge and after discarding the supernatant, re-suspended in 20 mL of DMF. Lysine membrane anchor—ε-hydrochloride (1.5 g; 1.65 mmol) was dissolved in dichloromethane (50 mL) and extracted twice with 100 mL of saturated sodium bicarbonate solution to remove the hydrochloride and free the ε-amine. After drying over sodium sulfate and filtration, the solution of the lysine membrane anchor was concentrated to 10 mL and added to the suspension of the p-nitrophenyl carbonate Wang resin. N-Methyl-morpholine (0.9 mL; 8 mmol) was added and the reaction was shaken for 24 hours on a Labquake shaker. The resin was then filtered and washed with DMF; then incubated 12 hours with methanol (30 mL) and 2 M DIPEA in NMP (20 mL) to endcap any unreacted resin sites. The resin was filtered and washed alternating with NMP and dichloromethane until the filtrate became colorless; then air dried, then under high vacuum (to 10 mT) to constant weight. The slightly yellow resin (1.33 g) was assayed for Fmoc using standard deprotection/UV assays. The load was determined to be 0.236 mmol/g; which corresponds to 50% of the theoretical loading. A sample of the resin was deprotected with TFA and the residue on evaporation gave a weight corresponding to the previously determined load of 0.24 mmol/g and analytical data by NMR in accordance to the lysine membrane anchor attached to the resin.

### EXAMPLE 2

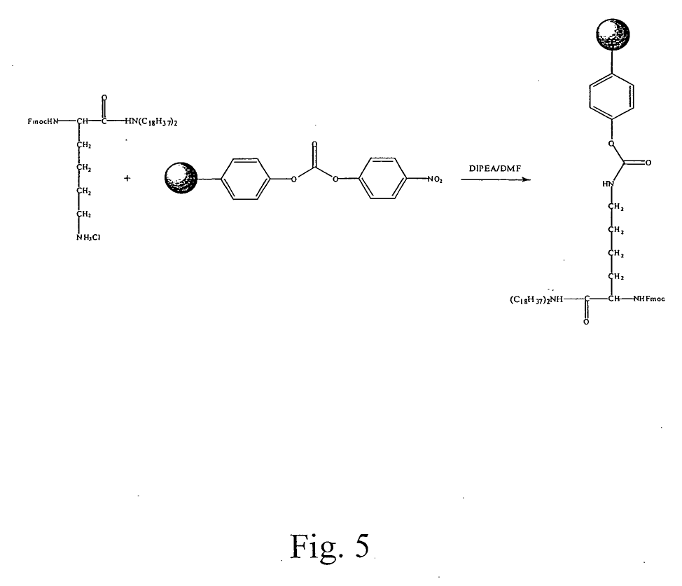

The immobilized Lysine membrane anchor on a p-nitrophenyl carbonate Wang resin (shown in FIG. 5) was used on an ABI 433A peptide synthesizer. Attachment efficiency for first residue was determined as follows. Using the standard FastMoc protocol on a 0.01 mmol scale, the conductivity of the Fmoc deprotection was monitored for consecutive runs of an initial attachment of a glycine residue. The average coupling yield was determined by Fmoc deprotection of the newly attached residue as well as TFA deprotection. Following purification, the weight was measured and NMR spectra taken. The coupling of the first residue was accomplished in 78% for single coupling and above 98% for double coupling of Fmoc Gly.

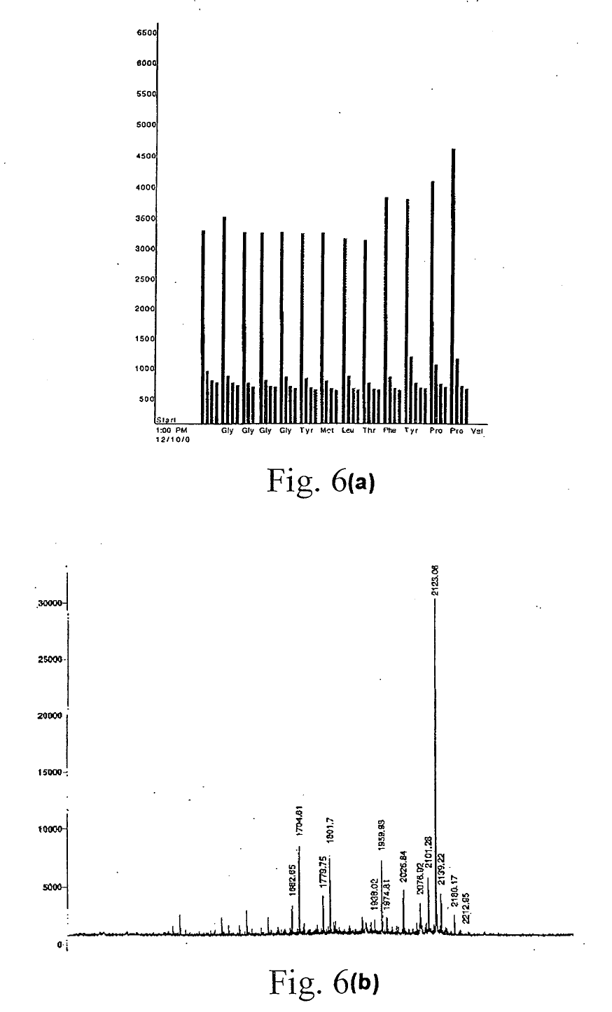

The immobilized Lysine membrane anchor on p-nitrophenyl carbonate Wang resin was further tested for peptide syntheses by generating a model peptide “VPPYFTLMYGGGGK” on the resin using the standard FastMoc protocol. The conductivity trace for the peptide synthesis is shown in FIG. 6(a) and demonstrated an efficient coupling of consecutive residues. FIG. 6(b) show a MALDI-MS for the peptide “VPPYFTLMYGGGGK” synthesized on the resin immobilized-membrane anchor. Deprotection with TFA on the resin sample was followed by MALDI-TOF-MS analysis (FIG. 6(b)) of the crude product and showed the signals expected for the fully deprotected peptide membrane anchor at (M 2122+1). More specifically, the identity, synthetic efficiency and “synthetic purity” of the peptide were confirmed by peptide hydrolysis (TFA) and the MALDI-MS analysis of the crude products. The MALDI analysis showed the expected peak distribution around M+ and other minor peaks due to partial deprotection, various protonation states due to the acidity of the deprotection/spotting procedure, and in addition some oxidation of part of the methionines to the sulfone, which was expected under these conditions. Note that the MALDI-MS showed a very narrow distribution of product peaks around M+, which confirmed the efficient use of this immobilized membrane anchor.

Although the present invention has been described with reference to specific details, it is not intended that such details should be regarded as limitations upon the scope of the invention, except as and to the extent that they are included in the accompanying claims.

What is claimed is:

1. A composition comprising a solid resin support having a trifunctional chemical moiety covalently attached thereto at a resin attachment site, said trifunctional chemical moiety including one or more hydrophobic membrane anchoring groups as one functionality thereon, said hydrophobic membrane anchoring groups containing from 4 to 30 carbon atoms with the proviso that said hydrophobic membrane anchoring groups are not directly attached to said solid resin support.

2. The composition of claim 1 wherein said hydrophobic membrane anchoring groups contain from 6 to 18 carbon atoms.

3. The composition of claim 1 wherein said trifunctional chemical moiety comprises an amino acid derivative including alkyl side chains thereon, and a reactive arm group selected from the group consisting of amino, carboxyl and sulfhydryl covalently attached to said resin attachment site.

4. The composition of claim 2 wherein said trifunctional chemical moiety comprises an amino acid derivative including alkyl side chains thereon, a reactive group selected from the group consisting of amino, hydroxyl, carboxyl and sulfhydryl thereon and a reactive arm group selected from the group consisting of amino, carboxyl and sulfhydryl covalently attached to said resin attachment site.

5. The composition of claim 3 wherein said reactive group includes a protecting group thereon.

6. The composition of claim 3 wherein said reactive arm group is an amino group covalently attached to said resin attachment site.

7. The composition of claim 3 wherein said reactive arm group is a carboxyl group covalently attached to said resin attachment site.

8. The composition of claim 3 wherein said amino acid is selected from the group consisting of glutamic acid, lysine, serine, homoserine, aspartic acid, and cysteine.

9. The composition of claim 3 wherein said protected reactive group includes a protecting group selected from the group consisting of Fmoc, and Boc.

10. The composition of claim 1 wherein said solid resin support is selected from the group consisting of ceramics, glass, latex, crosslinked polystyrenes, crosslinked polyacrylamides, natural polymers, gold, colloidal metal particles, silica gels, aerogels and hydrogels.

11. A method of solid phase peptide synthesis comprising
reacting a trifunctional chemical moiety comprising an amino acid derivative including one or more alkyl side chains having from 4 to 30 carbons as hydrophobic membrane anchoring groups thereon, a reactive group selected from the group consisting of amino, hydroxyl, carboxyl and sulfhydryl thereon and a reactive arm group selected from the group consisting of amino, carboxyl and sulfhydryl thereon with a solid resin support having a reactive site thereon to form a bound trifunctional chemical moiety-solid resin support composite; and,
reacting said bound trifunctional chemical moiety-solid resin support composite in a solid phase synthesis process.

12. The method of claim 11 wherein said solid resin support is selected from the group consisting of ceramics, glass, latex, crosslinked polystyrenes, crosslinked polyacrylamides, natural polymers, gold, colloidal metal particles, silica gels, aerogels and hydrogels.
